(19) United States
(12) Patent Application Publication
(10) Pub. No.: US 2004/0097734 A1
(43) Pub. Date: May 20, 2004

(54) Aryl- and heteroarylcarbonylpiperazines and their use for the treatment of benign and malignant oncoses

(76) Inventors: Matthias Gerlach, Brachtall (DE); Peter Emig, Bruchkobel (DE); Emmanuel Polymeropoulos, Frankfurt (DE); Gilbert Muller, Frankfurt (DE); Peter Schmidt, Schoneck (DE); Silke Baasner, Schoneck (DE); Eckhard Gunther, Maintal (DE)

Correspondence Address: GOODWIN PROCTER L.L.P, 103 EISENHOWER PARKWAY, ROSELAND, NJ, 07068, US

(21) Appl. No.: 10/608,520

(22) Filed: Jun. 27, 2003

Related U.S. Application Data
(60) Provisional application No. 60/393,027, filed on Jun. 29, 2002.

(51) Int. Cl. C 07D 4 3/02; C07D 417/02; C 07D 4 5/02

(52) U.S. Cl. 544/323; 544/235; 544/374; 544/380; 544/371; 544/375

(57) ABSTRACT

The invention relates to novel aryl- and heteroarylcarboxamides of the general formula (1), their preparation and use as medicaments, in particular for the treatment of tumors.

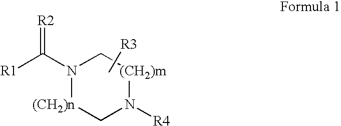

### CLAIM OF PRIORITY

This application claims the benefit of priority under 35 U.S.C. 119 of Provisional Application No. 60/393,027 filed on Jun. 29, 2002, the entire contents of which are incorporated in this application by reference thereto.

### DESCRIPTION

For the next few years, a dramatic increase in oncoses and tumor-related deaths is expected worldwide. In 2001, worldwide approximately 10 million people were suffering from cancer and over 6 million people died from this disease. The development of tumors is a fundamental disease of higher organisms in the plant kingdom, in the animal kingdom and in humans. The generally recognized multistep model of carcinogenesis assumes that as a result of the accumulation of a number of mutations in an individual cell it is so modified in its proliferation and differentiation behavior that finally, via benign intermediate stages, a malignant state with metastasis is reached. Behind the term cancer or tumor, a clinical picture with more than 200 various individual diseases hides itself. Oncoses can proceed in a benign or malignant manner. The most important tumors are those of the lung, the breast, the stomach, the neck of the uterus, the prostate, the head and neck, the large and small intestine, the liver and the blood system. There are great differences with respect to course, prognosis and therapy behavior. More than 90% of the cases recognized relate to solid tumors, which in particular in the advanced stage or on metastasis are treatable with difficulty or untreatable. The three pillars of cancer control are still surgical removal, irradiation and chemotherapy. In spite of great advances it has still not been possible to develop medicaments which bring about a marked prolongation of the survival time or even a complete cure in the widespread solid tumors. It is therefore meaningful to invent novel medicaments for the control of cancer.

The present invention relates to novel aryl- and heteroaryl-substituted piperazinylcarbonyls and their homologs, their preparation and use as medicaments, in particular for the treatment of benign and malignant tumors in humans and mammals.

For example, in the patent specifications WO 2002 008194, WO 2002 008192 and WO 2002 008190 of the company Zentaris AG substituted and unsubstituted acridine-, quinoline- or pyridinocarbonylpiperazides having anticarcinogenic properties are described.

In the patent specifications DE 1102747 and U.S. Pat. No. 3,843,657, fluorene derivatives having antispasmolytic or having antibacterial and fungicidal properties are described. A tumor action is neither described nor suggested.

Xanthene derivatives are described in the literature as antispasmolytics (U.S. Pat. No. 2,742,472) and antiulcer agents (U.S. Pat. No. 3,284,449). A tumor action is neither described nor suggested. Cinnoline derivatives of the abovementioned substance type are mentioned in the literature having different biological properties, for example as antiinflammatories (J. Med. Chem. 1966, 9, 664) or having CNS activity (A. Stanczak et al. Pharmazie 1997, 521, 91-97; U.S. Pat. No. 3,299,070). A tumor action is neither described nor suggested.

Isoquinoline derivatives and their use as local anesthetics are described by, F. Duro et al. in Farmaco, 1981, 36(6), 400-411. Moreover, isoquinolines of the abovementioned structural type are used as antipyretics, antiarrhythmics and sedatives (DE 2811312, DE 2818423). A tumor activity is neither described nor suggested.

Isoxazoles and isothiazoles are described in the patent specification U.S. Pat. No. 4,001,237 and by A. Carenzi et al. Arzneimittel Forsch. 1989, 39, 642 as potential antihypertensives. In addition, isoxazoles are described as fungicides (J. Heindl et al. Eur. J. of Med. Chem. 1975,10, 591). Isoxazoles are moreover confirmed in the literature as analgesics (DE2065430), muscarin receptor antagonists (H. g. Striegel et al. European J. of Med. Chem. 1995, 30, 839), having antibacterial properties (A. Pae et al. Biorg. Med. Chem. Left. 1999, 18, 2679). A tumor activity is neither described nor suggested.

Pyrazoles are mentioned in the literature as compounds having antiinflammatory and hypnotic properties (S. Sugiura et al. J. Med. Chem. 1977, 20, 80), as anxiolytics (J. K. Chakrabarti et al. J. Med. Chem. 1989, 32, 2573), having antibacterial properties (G. Palazzino et al. Farmaco Ed. Sci. 1986, 41, 566), as cannabinoid receptor antagonists (R. Lau et al. J. Med. Chem. 1999, 42, 769; R. Pertwee et al. Eur. J. Pharmacol. 1996, 296, 169), as alpha adrenoceptor antagonists (G. Ermandi et al. Farmaco Ed. Sci. 1998, 53, 519), as histamine H3 antagonists (WO2003004480), as factor Xa inhibitors (WO01/19798), as sedatives and analgesics (EP1006110), as cholinesterase inhibitors (WO98/39000) and as CRF receptor antagonists (U.S. Pat. No. 9,720,835). A tumor action is neither described nor suggested.

It has now surprisingly been found that novel compounds from the group consisting of the aryl- and heteroaryl-substituted piperazinylcarbonyl aromatics are suitable for the preparation of medicaments and these in particular are suitable for the treatment of benign and malignant tumors. According to this aspect, in the present application novel compounds from the group consisting of the aryl- and heteroaryl-substituted piperazinylcarbonyl compounds according to the general formula 1 are claimed,

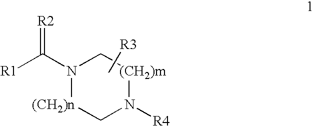

where the substituents have the following meaning:

R1: fluoren-9-one, isoxazole, cinnoline, isothiazole, isoquinoline, 9H-fluorene, 9H-xanthene and 1H-pyrazole,

where the bonding can take place via any desired and possible ring member of the heteroaryl or aryl radical and the aromatics and heteroaromatics can be mono- or polysubstituted or unsubstituted,

R2: O, S;

R3: represents one or up to 16 substituents selected from the group: H, unsubstituted or substituted alkyl, halogen, COOH, CONH₂, where the substituents can be arranged vicinally or geminally on the heterocycle;

R4: unsubstituted or substituted aryl, unsubstituted or substituted heteroaryl, unsubstituted or substituted alkylaryl, unsubstituted or substituted alkylhetaryl;

m, n: 0-3.

The expression “halogen” within the meaning of this invention comprises the halogen atoms fluorine, chlorine, bromine and iodine.

The expression “metal” within the meaning of this invention comprises metal ions such as sodium, potassium, lithium, magnesium, calcium, zinc and manganese ions.

The expression “alkyl” within the meaning of this invention comprises acyclic saturated or unsaturated hydrocarbon radicals, which can be branched or straight-chain and unsubstituted or mono- or polysubstituted, having 1 to 20 C atoms, i.e. C₁₋₂₀-alkanyls, C₂₋₂₀-alkenyls and C₂₋₂₀-alkynyls. In this context, alkenyls have at least one C—C double bond and alkynyls at least one C—C triple bond. Advantageously, alkyl is selected from the group which comprises methyl, ethyl, n-propyl, 2-propyl, n-butyl, sec-butyl, tert-butyl, n-pentyl, iso-pentyl, neopentyl, n-hexyl, 2-hexyl, n-octyl, ethylenyl(vinyl), ethynyl, propenyl(—CH₂CH═CH₂; —CH═CH—CH₃, —C(═CH₂)—CH₃), propynyl(—CH₂—C≡CH, —C≡C—CH₃), butenyl, butynyl, pentenyl, pentynyl, hexenyl, hexynyl, octenyl and octynyl.

The expression “cycloalkyl” for the purposes of this invention denotes cyclic hydrocarbons having 3-12 carbon atoms, which can be saturated or unsaturated, unsubstituted or substituted. The cycloalkyl radical can also be part of a bi- or polycyclic system.

The expression “heterocyclyl” stands for a 3-, 4-, 5-, 6-, 7- or 8-membered cyclic organic radical, which contains at least 1, optionally 2, 3, 4 or 5 heteroatoms, where the heteroatoms are identical or different and the cyclic radical is saturated or unsaturated, but not aromatic and can be unsubstituted or mono- or polysubstituted. The heterocycle can also be part of a bi- or polycyclic system. Preferred heteroatoms are nitrogen, oxygen and sulfur. It is preferred that the heterocyclyl radical is selected from the group which contains tetrahydrofuryl, tetrahydropyranyl, pyrrolidinyl, piperidinyl, piperazinyl and morpholinyl, where the bonding to the compound of the general formula 1 can take place via any desired ring member of the heterocyclyl radical.

The expression “aryl” within the meaning of this invention means aromatic hydrocarbons, inter alia phenyls, naphthyls and anthracenyls. The radicals can also be fused to further saturated, (partially) unsaturated or aromatic ring systems. Each aryl radical can be present in unsubstituted or mono- or polysubstituted form, where the aryl substituents can be identical or different and can be in any desired and possible position of the aryl.

The expression “heteroaryl” stands for a 5-, 6- or 7-membered cyclic aromatic radical, which contains at least 1, optionally also 2, 3, 4 or 5 heteroatoms, where the heteroatoms are identical or different and the heterocycle can be unsubstituted or mono- or polysubstituted; in the case of substitution on the heterocycle, the heteroaryl substituents are identical or different and are in any desired and possible position of the heteroaryl. The heterocycle can also be part of a bi- or polycyclic system. Preferred heteroatoms are nitrogen, oxygen and sulfur. It is preferred that the heteroaryl radical is selected from the group which contains pyrrolyl, furyl, thienyl, thiazolyl, triazolyl, tetrazolyl, oxazolyl, isothiazolyl, isoxazolyl, pyrazolyl, imidazolyl, pyridinyl, pyrimidinyl, pyrazinyl, triazinyl, benzothiazolyl, indolyl, indolizinyl, quinolinyl, isoquinolinyl, cinnolinyl, quinazolinyl, quinoxalinyl, phthalazinyl, carbazolyl, phenazinyl, phenothiazinyl, purinyl, acridinyl, phenanthrinyl, where the bonding to the compounds of the general formula 1 can take place via any desired and possible ring member of the heteroaryl radical.

The expressions “alkylcycloalkyl”, “alkylheterocyclyl”, “alkylaryl” or “alkylheteroaryl” mean for the purposes of the present invention that alkyl and cycloalkyl, heterocyclyl, aryl and heteroaryl have the meanings defined above and the cycloalkyl, heterocyclyl, aryl or heteroaryl radical is bonded via a C1-8-alkyl group to the compound of the general formula 1.

In connection with “alkyl”, “alkenyl” and “alkynyl”, the term substituted is understood within the meaning of this invention as meaning the substitution of a hydrogen radical by F, Cl, Br, I, CN, NH₂, NH-alkyl, NH-cycloalkyl, NH-aryl, NH-heteroaryl, NH-alkylaryl, NH-alkylheteroaryl, NH-heterocyclyl, NH-alkyl-OH, N(alkyl)₂, N(alkylaryl)₂, N(alkylheteroaryl)₂, N(heterocyclyl)₂, N(alkyl-OH)₂, NO, NO₂, SH, S-alkyl, S-cycloalkyl, S-aryl, S-heteroaryl, S-alkylaryl, S-alkylheteroaryl, S-heterocyclyl, S-alkyl-OH, S-alkyl-SH, S-alkyl, S—S-cycloalkyl, S—S-aryl, S—S-heteroaryl, S—S-alkylaryl, S—S-alkylheteroaryl, S—S-heterocyclyl, SS-alkyl-OH, S—S-alkyl-SH, S—S-alkyl-C(O)—NH-heterocyclyl, OH, O-alkyl, O-cycloalkyl, O-alkylcycloalkyl, O-aryl, O-heteroaryl, O-alkylaryl, O-alkylheteroaryl, O-heterocyclyl, O-alkylheterocyclyl, O-alkyl-OH, O-alkyl-O-alkyl, O—SO₂—N(alkyl)₂, O—SO₂—OH, O—SO₂—O-alkyl, O—SO₂—O-cycloalkyl, O—SO₂—O-heterocycloalkyl, O—SO₂—O-alkylcycloalkyl, O—SO₂—O-alkylheterocycloalkyl, O—SO₂—O-aryl, O—SO₂—O-heteroaryl, O—SO₂—O-alkylaryl, O—SO₂—O-alkylheteroaryl, O—SO₂-alkyl, O—SO₂-cycloalkyl, O—SO₂-heterocycloalkyl, O—SO₂-alkylcycloalkyl, O—SO₂-alkylheterocycloalkyl, O—SO₂-aryl, O—SO₂-heteroaryl, O—SO₂-alkylaryl, O—SO₂-alkylheteroaryl, O—C(O)-alkyl, O—C(O)-cycloalkyl, O—C(O)-heterocycloalkyl, O—C(O)-alkylcycloalkyl, O—C(O)-alkylheterocycloalkyl, O—C(O)-aryl, O—C(O)-heteroaryl, O—C(O)-alkylaryl, O—C(O)-alkylheteroaryl, O—C(O)O-alkyl, O—C(O)O-cycloalkyl, O—C(O)O-heterocycloalkyl, O—C(O)O-alkylcycloalkyl, O—C(O)O-alkylheterocycloalkyl, O—C(O)O-aryl, O—C(O)O-heteroaryl, O—C(O)O-alkylaryl, O—C(O)O-alkylheteroaryl, O—C(O)NH-alkyl, O—C(O)NH-cycloalkyl, O—C(O)NH-heterocycloalkyl, O—C(O)NH-alkylcycloalkyl, O—C(O)NH-alkylheterocycloalkyl, O—C(O)NH-aryl, O—C(O)NH-heteroaryl, O—C(O)NH-alkylaryl, O—C(O)NH-alkylheteroaryl, O—C(O)N(alkyl)₂, O—C(O)N(cycloalkyl)₂, O—C(O)N(heterocycloalkyl)₂, O—C(O)N(alkylcycloalkyl)₂, O—C(O)N(alkylheterocycloalkyl)₂, O—C(O)N(aryl)₂, O—C(O)N(heteroaryl)₂, O—C(O)N(alkylaryl)₂, O—C(O)N(alkylheteroaryl)₂, O—P(O)(OH)₂, O—P(O)(O-metal)₂, O—P(O)(O-cycloalkyl)₂, O—P(O)(O)-aryl)₂, O—P(O)(O-aryl)₂, O—P(O)(O-heteroaryl)₂, O—P(O)(O-alkylaryl)₂, O—P(O)(O)-alkylheteroaryl)₂, O—P(O)(N-alkyl)₂(N-alkyl)₂, O—P(O)(N-cycloalkyl)₂(N-cycloalkyl)₂, O—P(O)(N-heterocycloalkyl)₂(N-heterocycloalkyl)₂, O—P(O)(N-aryl)₂(N-aryl)₂, O—P(O)(N-heteroaryl)₂(N-heteroaryl)₂, O—P(O)(N-alkylaryl)₂(N-alkylaryl)₂, O—P(O)(N-alkylheteroaryl)₂(N-alkylheteroaryl)₂, CHO, C(O)-alkyl, C(S)-alkyl, C(O)-aryl, C(S)-aryl, C(O)-alkylaryl, C(S)-alkylaryl, C(O)-heterocyclyl, C(O)-heteroaryl, C(O)-alkylheteroaryl, C(S)-heterocyclyl, CO₂H, CO₂-alkyl, CO₂-cyclyl, CO₂-heterocyclyl, CO₂-aryl, CO₂-heteroaryl, CO₂-alkylaryl, C(O)NH-alkyl, C(O)NH-aryl, C(O)NH-heterocyclyl, C(O)NH-alkylheterocyclyl, C(O)N(alkyl)₂, C(O)N(alkylaryl)₂, C(O)N(alkylheteroaryl)₂, C(O)N(heterocyclyl)₂, SO-alkyl, SO₂-alkyl, SO₂-aryl, SO₂-alkylaryl, SO₂-heteroaryl, SO₂-alkylheteroaryl, SO₂NH₂, SO₃H, CF₃, CHO, CHS, alkyl, cycloalkyl, aryl, alkylaryl, heteroaryl, alkylheterocyclyl and/or heterocyclyl, where polysubstituted radicals are to be understood as meaning those which are either polysubstituted, e.g. di- or trisubstituted, on different or on identical atoms, for example trisubstituted on the same C atom as in the case of CF₃, —CH₂CF₃ or in different positions as in the case of —CH(OH)—CH═CH—CHCl₂. Polysubstitution can take place with the same or different substituents.

With respect to aryl, heterocyclyl, heteroaryl, alkylaryl and cycloalkyl, mono- or polysubstituted is understood within the meaning of this invention as meaning the mono- or polysubstitution, e.g. di-, tri- or tetrasubstitution, of one or more hydrogen atoms of the ring system by F, Cl, Br, I, CN, NH₂, NH-alkyl, NH-aryl, NH-heteroaryl, NH-alkylaryl, NH-alkylheteroaryl, NH-heterocyclyl, NH-alkyl-OH, N(alkyl)₂, NC(O)alkyl, N(alkylaryl)₂, N(alkylheteroaryl)₂, N(heterocyclyl)₂, N(alkyl-OH)₂, NO, NO₂, SH, S-alkyl, S-aryl, S-heteroaryl, S-alkylaryl, S-alkylheteroaryl, S-heterocyclyl, S-alkyl-OH, S-alkyl-SH, OH, O-alkyl, O-cycloalkyl, O-alkylcycloalkyl, O-aryl, O-heteroaryl, O-alkylaryl, O-alkylheteroaryl, O-heterocyclyl, O-alkylheterocyclyl, O-alkyl-OH, O-alkyl-O-alkyl, O—SO₂—N(alkyl)₂, O—SO₂—OH, O—SO₂—O-alkyl, O—SO₂—O-cycloalkyl, O—SO₂—O-heterocycloalkyl, O—SO₂—alkylcycloalkyl, O—SO₂—O-alkylheterocycloalkyl, O—SO₂—O-aryl, O—SO₂—O-heteroaryl, O—SO₂—O-alkylaryl, O—SO₂—O-alkylheteroaryl, O—SO₂-alkyl, O—SO₂-cycloalkyl, O—SO₂-heterocycloalkyl, O—SO₂-alkylcycloalkyl, O—SO₂-alkylheterocycloalkyl, O—SO₂-aryl, O—SO₂-heteroaryl, O—SO₂-alkylaryl, O—SO₂-alkylheteroaryl, O—C(O)-alkyl, O—C(O)-cycloalkyl, O—C(O)-heterocycloalkyl, O—C(O)—alkylcycloalkyl, O—C(O)-alkylheterocycloalkyl, O—C(O)-aryl, O—C(O)-heteroaryl, O—C(O)-alkylaryl, O—C(O)-alkylheteroaryl, O—C(O)O-alkyl, O—C(O)O-cycloalkyl, O—C(O)O-heterocycloalkyl, O—C(O)O-alkylcycloalkyl, O—C(O)O-alkylheterocycloalkyl, O—C(O)O-aryl, O—C(O)O-heteroaryl, O—C(O)O-alkylaryl, O—C(O)O-alkylheteroaryl, O—C(O)NH-alkyl, O—C(O)NH-cycloalkyl, O—C(O)NH-heterocycloalkyl, O—C(O)NH-alkylcycloalkyl, O—C(O)NH-alkylheterocycloalkyl, O—C(O)NH-aryl, O—C(O)NH-heteroaryl, O—C(O)NH-alkylaryl, O—C(O)NH-alkylheteroaryl, O—C(O)N(alkyl)₂, O—C(O)N(cycloalkyl)₂, O—C(O)N(heterocycloalkyl)₂, O—C(O)N(alkylcycloalkyl)₂, O—C(O)N(alkylheterocycloalkyl)₂, O—C(O)N(aryl)₂, O—C(O)N(heteroaryl)₂, O—C(O)N(alkylaryl)₂, O—C(O)N(alkylheteroaryl)₂, O—P(O)(OH)₂, O—P(O)(O-metal)₂, O—P(O)(O-alkyl)₂, O—P(O)(O-cycloalkyl)₂, O—P(O)(O-aryl)₂, O—P(O)(O-heteroaryl)₂, O—P(O)(O-alkylaryl)₂, O—P(O)(O-alkylheteroaryl)₂, O—P(O)(N-alkyl)₂(N-alkyl)₂, O—P(O)(N-cycloalkyl)₂(N-cycloalkyl)₂, O—P(O)(N-heterocycloalkyl)₂(N-heterocycloalkyl)₂, O—P(O)(N-aryl)₂(N-aryl)₂, O—P(O)(N-heteroaryl)₂(N-heteroaryl)₂, O—P(O)(N-alkylaryl)₂(N-alkylaryl)₂, O—P(O)(N-alkylheteroaryl)₂(N-alkylheteroaryl)₂, CHO, C(O)-alkyl, C(S)-alkyl, C(O)-aryl, C(S)-aryl, C(O)-alkylaryl, C(S)-alkylaryl, C(O)-heterocyclyl, C(S)-heterocyclyl, CO₂H, CO₂-alkyl, CO₂-alkylaryl, C(O)—NH₂, C(O)NH-alkyl, C(O)NH-aryl, C(O)NH-heterocyclyl, C(O)N(alkyl)₂, C(O)N(alkylaryl)₂, C(O)N(alkylheteroaryl)₂; C(O)N(heterocyclyl)₂, SO-alkyl, SO₂-alkyl, SO₂-aryl, SO₂-alkylaryl, SO₂-heteroaryl, SO₂-alkylheteroaryl, SO₂NH₂CHO, CHS, alkyl, cycloalkyl, aryl, alkylaryl, heteroaryl, alkylheterocyclyl and/or heterocyclyl, on one or optionally different atoms (where one substituent can optionally for its part be substituted). Polysubstitution in this case takes place with the same or with different substituents.

If the compounds of the general formula 1 according to the invention have at least one asymmetric center, they can be present in the form of their racemates, in the form of the pure enantiomers and/or diastereomers or in the form of mixtures of these enantiomers and/or diastereomers. The mixtures can be present in any desired mixing ratio of the stereoisomers.

If possible, the compounds according to the invention can be present in the form of the tautomers.

Thus, for example, the compounds according to the invention as in the general formula 1, which have one or more chiral centres and which occur as racemates, can be separated into their optical isomers, that is enantiomers or diastereomers, by methods known per se. The separation can be carried out by column separation on chiral phases or by recrystallization from an optically active solvent or using an optically active acid or base or by derivativization with an optically active reagent, such as, for example, an optically active alcohol, and subsequent removal of the radical.

The compounds of the general formula 1 according to the invention can, if they have a sufficiently basic group, such as, for example, a secondary or tertiary amine, be converted into salts using inorganic and organic acids. Preferably, the pharmaceutically acceptable salts of the compounds according to the mention as in the general structure 1 with hydrochloric acid, hydrobromic acid, sulfuric acid, phosphoric acid, methanesulfonic acid, p-toluenesulfonic acid, carbonic acid, formic acid, acetic acid, sulfoacetic acid, trifluoroacetic acid, oxalic acid, malonic acid, maleic acid, succinic acid, tartaric acid, racemic acid, malic acid, embonic acid, mandelic acid, fumaric acid, lactic acid, citric acid, taurocholic acid, glutamic acid or aspartic acid are formed. The salts formed are, inter alia, hydrochlorides, hydrobromides, sulfates, phosphates, methanesulfonates, tosylates, carbonates, hydrogencarbonates, formates, acetates, sulfoacetates, triflates, oxalates, malonates, maleates, succinates, tartrates, malates, embonates, mandelates, fumarates, lactates, citrates and glutamates. The stoichiometry of the salts of the compounds according to the invention formed can in this case be an integral or nonintegral multiple of one.

The compounds of the general formula 1 according to the invention can, if they contain a sufficiently acidic group, such as, for example, the carboxyl group, sulfonic acid, phosphoric acid or a phenolic group, be converted into their physiologically tolerable salts with inorganic and organic bases. Possible inorganic bases are, for example, sodium hydroxide, potassium hydroxide, calcium hydroxide, as organic bases ethanolamine, diethanolamine, triethanolamine, cyclohexylamine, dibenzylethylenediamine and lysine. The stoichiometry of the salts of the compounds according to the invention formed can in this context be an integral or nonintegral multiple of one.

Likewise preferred are solvates and in particular hydrates of the compounds according to the invention, which can be obtained, for example, by crystallization from a solvent or from aqueous solution. In this context, one, two, three or as many solvate or water molecules as liked can be combined with the compounds according to the invention to give solvates and hydrates.

It is known that chemical substances form solids which are present in various atomic states, which are described as polymorphic forms or modifications. The different modifications of a polymorphic substance can differ greatly in its physical properties. The compounds of the general formula 1 according to the invention can be present in various polymorphic forms, in this context certain modifications can be metastable.

According to a further embodiment, the compounds according to the invention as in the general formula 1 are made available, wherein R₁, R₂, R₃, n and m have the abovementioned meanings and R₄ stands for phenyl which is unsubstituted or substituted by one to five identical or different (C₁-C₆)-alkoxy groups, where adjacent oxygen atoms can also be linked by (C₁-C₂)-alkylene groups.

According to a further embodiment, compounds according to the general formula 1 are made available, wherein R, R₁, R₂, R₃, n and m have the abovementioned meanings and R₄ stands for 3,5-dimethoxyphenyl.

According to a further embodiment, compounds according to the general formula 1 are made available, wherein R₁, R₂, R₃, n and m have the abovementioned meanings and R₄ stands for 3-methoxyphenyl.

Most preferred are compounds according to the general formula 1, which are found in the following selection:

4-[4-(3,5-Dimethoxyphenyl)piperazine-1-carbonyl]fluoren-9-one (1)

4-[4-(6-Methylpyridin-2-yl)piperazine-1-carbonyl]fluoren-9-one (2)

4-[4-(3-Hydroxyphenyl)piperazine-1-carbonyl]fluoren-9-one (3)

[4-(3,5-Dimethoxyphenyl)piperazin-1-yl]-(5-methyl-3-phenylisoxazol-4-yl)methanone (4)

Cinnolin-4-yl-[4-(3,5-dimethylphenyl)piperazin-1-yl]methanone (5)

Cinnolin-4-yl-[4-(6-methylpyridin-2-yl)piperazin-1-yl]methanone (6)

(3,5-Bismethylsulfanylisothiazol-4-yl)-[4-(6-methylpyridin-2-yl)piperazin-1-yl]methanone (7)

[4-(3,5-Dimethoxyphenyl)piperazin-1-yl]isoquinolin-1-ylmethanone (8)

[4-(3,5-Dimethoxyphenyl)piperazin-1-yl]-(9H-fluoren-1-yl)methanone (9)

(9H-Fluoren-9-yl)-[4-(3-methoxyphenyl)piperazin-1-yl]methanone (10)

(9H-Fluoren-1-yl)-[4-(3-methoxyphenyl)piperazin-1-yl]methanone (11)

[4-(3,5-Dimethoxyphenyl)piperazin-1-yl]-(9H-xanthen-9-yl)methanone (12)

[4-(3-Methoxyphenyl)piperazin-1-yl]-(9H-xanthen-9-yl)methanone (13)

[4-(3-Methoxyphenyl)piperazin-1-yl]-(2-phenyl-2H-pyrazol-3-yl)methanone (14)

[4-(6-Methylpyridin-2-yl)piperazin-1-yl]-(2-phenyl-2H-pyrazol-3-yl)methanone (15)

[4-(3-Hydroxyphenyl)piperazin-1-yl]-(2-phenyl-2H-pyrazol-3-yl)methanone (16)

[4-(3,5-Dimethoxyphenyl)piperazin-1-yl]-[1-(4-nitrophenyl)-5-trifluoromethyl-1H-pyrazol-4-yl]-methanone (17)

According to a further aspect of the invention, a process for the preparation of the compounds according to the invention is claimed, which comprises reacting a carboxylic acid derivative of the general formula 2, in which R₁ and R₂ have the abovementioned meanings and Y represents a leaving group such as halogen, hydroxyl, (C₁-C₆)-alkoxy, preferably methoxy and ethoxy, —O-tosyl, —O-mesyl, tetrazolyl or imidazolyl,

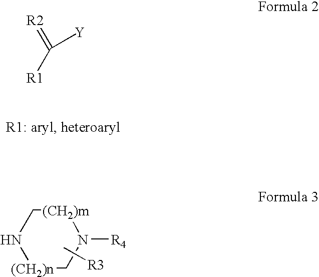

with an amine of the general formula 3, in which R₄, m and n have the above-mentioned meanings, optionally using a condensing agent and/or catalyst, and also diluents and auxiliaries with formation of the desired product as in the general formula 1.

Synthesis of the Compounds According to the Invention

The compounds of the general formula 1 are obtainable, for example, as in scheme 1 below:

Scheme 1

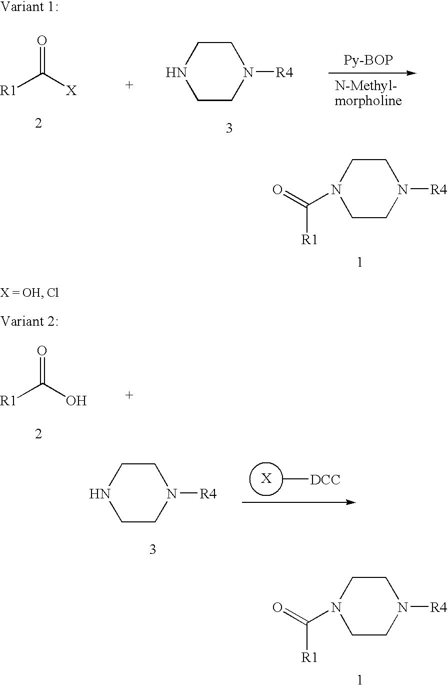

The starting compounds 2 and 3 are either commercially obtainable or can be prepared by procedures known per se. The starting materials 2 and 3 are valuable intermediate compounds for the preparation of the compounds of the formula 1 according to the invention.

The solvents and auxiliaries optionally to be used and reaction parameters such as reaction temperature and time to be used are known to the person skilled in the art on account of his/her expert knowledge.

The compounds according to the invention as in the general formula 1 are suitable as active compounds in the medicaments, in particular as antitumor agents, for the treatment of humans and mammals. Mammals can be domestic animals such as horses, cows, dogs, cats, hares, sheep and the like.

The medicinal action of the compounds according to the invention can be based, for example on an interaction with the tubulin system by inhibition of tubulin polymerization. In addition, still further known and unknown mechanisms of action for the control of tumor cells are conceivable.

According to a further aspect of the invention, a process for the control of tumors in humans and in mammals is made available; which comprises administering at least one compound according to the invention as in the general formula 1 to the human or a mammal in an amount effective for tumor treatment. The therapeutically effective dose of the respective compound according to the invention to be administered for the treatment depends, inter alia, on the nature and the stage of the oncosis, the age and sex of the patient, the manner of administration and the duration of treatment. The medicaments according to the invention can be administered as liquid, semisolid and solid pharmaceutical forms. This is carried out in the manner suitable in each case in the form of aerosols, powders and dusting powders, tablets, coated tablets, emulsions, foams, solutions, suspensions, gels, ointments, pastes, pills, pastels, capsules or suppositories.

The pharmaceutical forms contain, in addition to at least one constituent according to the invention, depending on the pharmaceutical form employed, optionally excipients, such as, inter alia, solvents, solution accelerators, solubilizers, emulsifiers, wetting agents, antifoams, gel-forming agents, thickeners, film-forming agents, binders, buffers, salt-forming agents, drying agents, flow regulators, fillers, preservatives, antioxidants, colorants, mold release agents, lubricants, disintegrants, taste and odor corrigents. The selection of the excipients and the amounts thereof to be employed depends on the chosen pharmaceutical form and is orientated to the recipes known to the person skilled in the art.

The medicaments according to the invention can be administered in a suitable administration form to the skin, epicutaneously as a solution, suspension, emulsion, foam, ointment, paste or patch; via the oral and lingual mucosa, buccally, lingually or sublinguaily as a tablet, pastille, coated tablets, linctus or gargle; via the gastric and intestinal mucosa, enterally as a tablet, coated tablets, capsule, solution, suspension or emulsion; via the rectal mucosa, rectally as a suppository, rectal capsule or ointment; via the nasal mucosa, nasally as drops, ointments or spray; via the bronchial and alveolar epithelium, pulmonarily or by inhalation as an aerosol or inhalate; via the conjunctiva, conjunctivally as eyedrops, eye ointment, eye tablets, lamellae or eye lotion; via the mucosa of the genital organs, intravaginally as vaginal suppositories, ointments and flush, intrauterinely as a uterine pessary; via the efferent ureters, intraurethrally as a flush, ointment or medicated sound; into an artery, intraarterially as an injection; into a vein, intravenously as an injection or infusion, paravenously as an injection or infusion; into the skin, intracutaneously as an injection or implant; under the skin, subcutaneously as an injection or implant; into the muscle, intramuscularly as an injection or implant; into the abdominal cavity, intraperitoneally as an injection or infusion.

The compounds of the general structure 1 according to the invention can be retarded in their pharmaceutical action with respect to practical therapeutic requirements by means of suitable measures. This aim can be achieved in a chemical and/or pharmaceutical way. Examples of the achievement of a prolongation of action are the use of implants, liposomes, sustained release forms, nanoparticle suspensions and “prodrugs” of the compounds according to the invention, the formation of poorly soluble salts and complexes or the use of crystal suspensions.

The compounds of the general structure 1 according to the invention can be employed as an individual substance or in combination with further cytotoxic substances, such as, for example, cisplatin, carboplatin, doxorubicin, ifosfamide, cyclophosphamide, 5-FU, methotrexate or in combination with immunomodulators or antibodies and in particular in combination with inhibitors of signal transduction, such as, for example, herceptin, glivec or iressa.

Particularly preferred medicaments in this context are those which contain at least one compound from the following group of the compounds according to the invention:

4-[4-(3,5-Dimethoxyphenyl)piperazine-1-carbonyl]fluoren-9-one (1)

4-[4-(6-methylpyridin-2-yl)piperazine-1-carbonyl]fluoren-9-one (2)

4-[4-(3-Hydroxyphenyl)piperazine-1-carbonyl]fluoren-9-one (3)

[4-(3,5-Dimethoxyphenyl)piperazin-1-yl]-(5-methyl-3-phenylisoxazol-4-yl)methanone (4)

cinnolin-4-yl-[4-(3,5-dimethylphenyl)piperazin-1-yl]methanone (5)

cinnolin-4-yl-[4-(6-methylpyridin-2-yl)piperazin-1-yl]methanone (6)

(3,5-Bis-methylsulfanylisothiazol-4-yl)-[4-(6-methylpyridin-2-yl)piperazin-1-yl]methanone (7)

[4-(3,5-Dimethoxyphenyl)piperazin-1-yl]-isoquinolin-1-ylmethanone (8)

[4-(3,5-Dimethoxyphenyl)piperazin-1-yl]-(9H-fluoren-1-yl)methanone (9)

(9H-Fluoren-9-yl)-[4-(3-methoxyphenyl)piperazin-(1-yl]methanone (10)

(9H-Fluoren-1-yl)-[4-(3-methoxyphenyl)piperazin-1-yl]methanone (11)

[4-(3,5-Dimethoxyphenyl)piperazin-1-yl]-(9H-xanthen-9-yl)methanone (12)

[4-(3-methoxyphenyl)piperazin-1-yl]-(9H-xanthen-9-yl)methanone (13)

[4-(3-methoxyphenyl)piperazin-1-yl](2-phenyl-2H-pyrazol-3-yl)methanone (14)

[4-(6-methylpyridin-2-yl)piperazin-1-yl]-(2-phenyl-2H-pyrazol-3-yl)methanone (15)

[4-(3-Hydroxyphenyl)piperazin-1-yl]-(2-phenyl-2H-pyrazol-3-yl)methanone (16)

[4-(3,5-Dimethoxyphenyl)piperazin-1-yl]-[1-(4-nitrophenyl)-5-trifluoromethyl-1H-pyrazol-4-yl]-methanone (17)

and can be present both as a free base and as salts of physiologically tolerable acids.

According to this general procedure, on which synthesis scheme 1 is based, the following compounds were synthesized which follow from the list below with statement of the respective chemical name. The analytical characterization of the compounds according to the invention was carried out by means of their melting points or by ¹H-NMR spectroscopy and/or mass spectrometry.

The chemicals and solvents employed were obtained commercially from the conventional suppliers (Acros, Avocado, Aldrich, Fluka, Lancaster, Maybridge, Merck, Sigma, TCI etc.) or synthesized.

The invention is intended to be illustrated in greater detail with the aid of the following examples, without being restricted thereto.

### EXAMPLE 1

### Reaction as in Scheme 1, Variant 1

4-[4-(3,5-Dimethoxyphenyl)piperazine-1-carbonyl]fluoren-9-one (1)

A solution of 1 g (4.12 mmol) of 9-fluorenone-4-carbonyl chloride in 30 ml of dimethylformamide was treated successively with 0.67 g (6.59 mmol) of N-methylmorpholine, 0.92 g (4.12 mmol) of 1-(3,5-dimethoxyphenyl)piperazine and 2.36 g (4.53 mmol) of Py-BOP (1-benzotriazolyltripyrrolidinophosphonium hexafluorophosphate). The mixture was stirred for 12 hours at room temperature, allowed to stand overnight at room temperature, dimethylformamide was distilled off in vacuo and the residue was purified through a silica gel column (silica gel 60, from Merck AG, Darmstadt) using the eluent dichloromethane/methanol (95:5 v/v).

Yield: 1.4 g (79.3% of theory)

M.p.: 161° C.

¹H-NMR (DMSO-d6) δ=7.71-7.4 (m, 7H), 6.08 (s, 2H), 6.0 (s, 1H), 3.98-3.85 (m, 2H), 3.68 (s, 6H), 3.45-2.9 (m, 6H) ppm.

### EXAMPLE 2

### Reaction as in Scheme 1, Variant 1

[4-(3,5-Dimethoxyphenyl)piperazin-1-yl]-(9H-xanthen-9-yl)methanone (12)

A solution of 3 g (13.26 mmol) of xanthene-9-carboxylic acid in 90 ml of dimethylformamide was treated successively with 2.15 g (21.2 mmol) of N-methylmorpholine, 2.95 g (13.26 mmol) of 1-(3,5-dimethoxyphenyl)piperazine and 7.59 g (14.59 mmol) of Py-BOP (1-benzotriazolyltripyrrolidinophosphonium hexafluorophosphate). The mixture was stirred for 12 hours at room temperature, allowed to stand overnight at room temperature, dimethylformamide was distilled off in vacuo and the residue was purified through a silica gel column (silica gel 60, from Merck AG, Darmstadt) using the eluent dichloromethane/methanol (95:5 v/v).

Yield: 2.88 g (50.4% of theory)

M.p.: 155° C.

¹H-NMR (DMSO-d6) δ=7.28 (d, 2H), 7.23 (d, 2H), 7.15 (d, 2H), 7.07 (t, 2H), 6.12 (s, 2H), 6.03 (s, 1H), 5.72 (s, 1H), 4.03 (m, 2H), 3.71 (s, 6H), 3.58 (m, 2H), 3.23-3.06 (m, 4H) ppm.

### EXAMPLE 3

### Reaction as in Scheme 1, Variant 2

[4-(3-Methoxyphenyl)piperazin-1-yl]-(2-phenyl-2H-pyrazol-3-yl)methanone (14)

A solution of 3.03 g (16.1 mmol) of 1-phenyl-1H-pyrazole-5-carboxylic acid in 40 ml of dimethylformamide was treated with 13.56 g (25.76 mmol) of polymer-bound N-benzoyl-N-cyclohexylcarbodiimide (1.66 mmol/g), warmed to 60° C. and the components were reacted with one another for 30 minutes. For this, 2.48 g (12.88 mmol) of 1-(3-methoxyphenyl)piperazine were added and the mixture was allowed to react for a further 4 hours. After this, it was allowed to cool, the resin was separated off, the dimethylformamide was distilled off in vacuo and the residue was purified through a silica gel column (silica gel 60, from Merck AG, Darmstadt) using the eluent dichloromethane/methanol (95:5 v/v).

Yield: 0.75 g (12.6% of theory)

¹H-NMR (DMSO-d6) δ=7.82 (s, 1H), 7.54-7.46 (m, 4H), 7.4 (t, 1H), 7.11 (t, 1H), 6.73 (d, 1H), 6.46 (m, 1H), 6.41-6.38 (m, 2H), 3.72 (m, 5H), 3.33 (m, 2H), 3.10 (m, 2H), 2.82 (m, 2H) ppm.

The following compounds of the general formula 1 were synthesized analogously to the synthesis route (variant 1 or 2) in scheme 1:

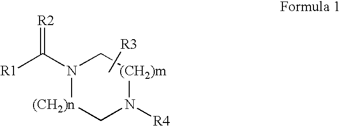

### EXAMPLE 4

### (4-[4-(6-Methylpyridin-2-yl)piperazine-1-carbonyl]fluoren-9-one (2)

¹H-NMR (DMSO-d6) δ=7.72 (d, 1H), 7.68 (d, 1H), 7.62 (t, 1H), 7.54 (d, 1H), 7.51-7.40 (m, 4H), 6.6 (d, 1H), 6.55 (d, 1H), 3.95 (m, 1H), 3.87 (m, 1H), 3.7 (m, 2H), 3.52-3.25 (m, 4H), 2.28 (s, 3H) ppm.

### EXAMPLE 5

### (4-[4-(3-Hydroxyphenyl)piperazine-1-carbonyl]fluoren-9-one (3)

ESI-MS: 385.1 [M+H]

### EXAMPLE 6

### [4-(3,5-Dimethoxyphenyl)piperazin-1-yl]-(5-methyl-3-phenylisoxazol-4-yl)methanone (4)

¹H-NMR (DMSO-d6) δ=7.58 (m, 2H), 7.47 (m, 3H), 5.96 (m, 3H), 3.75-3.63 (m, 8H), 3.26 (m, 4H), 3.15 (m, 2H), 2.48 (s, 3H) ppm.

### EXAMPLE 7

### Cinnolin-4-yl-[4-(3,5-dimethylphenyl)piperazin-1-yl]methanone (5)

M.p.: 114° C.

¹H-NMR (DMSO-d6) δ=9.45 (s, 1H), 8.58 (d, 1H), 8.04 (m, 1H), 7.96 (m, 2H), 6.58 (s, 2H), 6.48 (s, 1H), 3.95 (m, 2H), 3.34 (m, 2H), 3.28 (m, 2H), 3.05 (m, 2H), 2.21 (s, 6H) ppm.

### EXAMPLE 8

### Cinnolin-4-yl-[4-(6-methylpyridin-2-yl)piperazin-1-yl]methanone (6)

¹H-NMR (DMSO-d6) δ=9.43 (s, 1H), 8.58 (d, 1H), 8.05 (m, 1H), 7.95 (m, 2H), 7.45 (t, 1H), 6.63 (d, 1H), 6.54 (d, 1H), 3.90 (m, 2H), 3.72 (m, 2H), 3.48-3.2 (m, 4H), 2.3 (s, 3H) ppm.

### EXAMPLE 9

### (3,5-Bismethylsulfanylisothiazol-4-yl)-[4-(6-methylpyridin-2-yl)piperazin-1-yl]methanone (7)

¹H-NMR (DMSO-d6) δ=7.45 (t, 1H); 6.65 (d, 1H), 6.57 (d, 1H), 3.8-3.3 (m, 8H), 2.66 (s, 3H), 2.58 (s, 3H), 2.32 (s, 3H) ppm.

### EXAMPLE 10

### [4-(3,5-Dimethoxyphenyl)piperazin-1-yl]isoquinolin-1-ylmethanone (8)

¹H-NMR (DMSO-d6) δ=8.54 (d, 1H), 8.06 (d, 1H), 7.98 (d, 1H), 7.92 (d, 1H), 7.83 (t, 1H), 7.72 (t, 1H), 6.08 (s, 2H), 5.99 (s, 1H), 3.95 (m, 2H), 3.68 (s, 6H), 3.35 (m, 2H), 3.24 (m, 2H), 3.05 (m, 2H) ppm.

### EXAMPLE 11

### [4-(3,5-Dimethoxyphenyl)piperazin-1-yl]-(9H-fluoren-1-yl)methanone (9)

M.p.: 148° C.

¹H-NMR (DMSO-d6) δ=7.98 (d, 2H), 7.94 (d, 2H), 7.58 (d, 1H), 7.48 (t, 1H), 7.4 (t, 1H), 7.35 (t, 1H), 7.28 (d, 1H), 6.10 (s, 2H), 5.99 (s, 1H), 3.88 (s, 2H), 3.82 (m, 2H), 3.67 (s, 6H), 3.41 (m, 2H), 3.28 (m, 2H), 3.08 (m, 2H) ppm.

### EXAMPLE 12

### (9H-Fluoren-9-yl)-[4-(3-methoxyphenyl)piperazin-1-yl]methanone (10)

M.p.: 162-163° C.

¹H-NMR (DMSO-d6) δ=7.86 (d, 2H), 7.37 (d, 2H), 7.32 (t, 2H), 7.22 (t, 2H), 7.03 (t, 1H), 6.46 (m, 1H), 6.38 (s, 1H), 6.30 (d, 1H), 5.32 (s, 1H), 3.95-3.42 (m, 7H), 3.25-3.0 (m, 4H) ppm.

### EXAMPLE 13

### (9H-Fluoren-1-yl)-[4-(3-methoxyphenyl)piperazin-1-yl]methanone (11)

M.p.: 124° C.

¹H-NMR (DMSO-d6) δ=7.99 (d, 1H), 7.96 (d, 1H), 7.61 (d, 1H9, 7.48 (t, 1H), 7.42 (t, 1H), 7.35 (t, 1H), 7.29 (d, 1H), 7.12 (t, 1H), 6.54 (m, 1H), 6.48 (s, 1H), 6.39 (m, 1H), 3.89 (s, 2H), 3.83 (m, 2H), 3.71 (s, 3H), 3.41 (m, 2H), 3.27 (m, 2H), 3.08 (m, 2H) ppm.

### EXAMPLE 14

### [4-(3-Methoxyphenyl)piperazin-1-yl]-(9H-xanthen-9-yl)methanone (13)

M.p.: 110° C.

¹H-NMR (DMSO-d6) δ=7.30 (t, 2H), 7.22 (t, 2H), 7.15-7.05 (m, 5H), 6.56 (d, 1H), 6.4 (d, 1H), 5.74 (s, 1H), 4.05 (m, 2H), 3.74 (s, 3H), 3.58 (m, 2H), 3.2-3.06 (m, 4H) ppm.

### EXAMPLE 15

### [4-(6-Methylpyridin-2-yl)piperazin-1-yl]-(2-phenyl-2H-pyrazol-3-yl)methanone (15)

¹H-NMR (DMSO-d6) δ=7.83 (s, 1H), 7.55-7.37 (m, 6H), 6.74 (d, 1H), 6.57 (d, 1H), 6.53 (d 1H), 3.68 (m, 2H), 3.48 (m, 2H), 3.32 (m, 2H), 3.18 (m, 2H), 2.32 (s, 3H) ppm.

### EXAMPLE 16

### [4-(3-Hydroxyphenyl)piperazin-1-yl]-(2-phenyl-2H-pyrazol-3-yl)methanone (16)

¹H-NMR (DMSO-d6) δ=9.2 (s, 1H), 7.82 (d, 1H), 7.53-7.46 (m, 4H), 7.4 (t, 1H), 6.73 (d, 1H), 6.33 (m, 1H), 6.23 (m, 2H), 3.68 (m, 2H), 3.35 (m, 2H), 3.05 (m, 2H), 2.75 (m, 2H) ppm.

### EXAMPLE 17

### [4-(3,5-Dimethoxyphenyl)piperazin-1-yl]-[1-(4-nitrophenyl)-5-trifluoromethyl-1H-pyrazol-4-yl]methanone (17)

¹H-NMR (DMSO-d6) δ=8.45 (d, 2H), 8.18 (s, 1H), 7.88 (d, 2H), 6.1 (s, 2H), 6.0 (s, 1H) 3.77 (m, 2H), 3.69 (s, 6H), 3.53 (m, 2H), 3.2 (m, 2H), 3.12 (m, 2H) ppm.

The most preferred compounds of the present invention are substances of the general formula 1 in the form of their bases or their pharmaceutically acceptable salts, which are selected from the following group:

4-[4-(3,5-Dimethoxyphenyl)piperazine-1-carbonyl]fluoren-9-one (1)

4-[4-(6-methylpyridin-2-yl)piperazine-1-carbonyl]fluoren-9-one (2)

4-[4-(3-Hydroxyphenyl)piperazine-1-carbonyl]fluoren-9-one (3)

[4-(3,5-Dimethoxyphenyl)piperazin-1-yl]-(5-methyl-3-phenylisoxazol-4-yl)methanone (4)

Cinnolin-4-yl-[4-(3,5-dimethylphenyl)piperazin-1-yl]methanone (5)

Cinnolin-4-yl-[4-(6-methylpyridin-2-yl)piperazin-1-yl]methanone (6)

(3,5-Bismethylsulfanylisothiazol-4-yl)-[4-(6-methylpyridin-2-yl)piperazin-1-yl]methanone (7)

[4-(3,5-Dimethoxyphenyl)piperazin-1-yl]-isoquinolin-1-ylmethanone (8)

[4-(3,5-Dimethoxyphenyl)piperazin-1-yl]-(9H-fluoren-1-yl)methanone (9)

(9H-Fluoren-9-yl)-[4-(3-methoxyphenyl)piperazin-1-yl]methanone (10)

(9H-Fluoren-1-yl)-[4-(3-methoxyphenyl)piperazin-1-yl]methanone (11)

[4-(3,5-Dimethoxyphenyl)piperazin-1-yl]-(9H-xanthen-9-yl)methanone (12)

[4-(3-Methoxyphenyl)piperazin-1-yl]-(9H-xanthen-9-yl)methanone (13)

[4-(3-Methoxyphenyl)piperazin-1-yl]-(2-phenyl-2H-pyrazol-3-yl)methanone (14)

[4-(6-Methylpyridin-2-yl)piperazin-1-yl]-(2-phenyl-72H-pyrazol-3-yl)methanone (15)

[4-(3-Hydroxyphenyl)piperazin-1-yl]-(2-phenyl-2H-pyrazol-3-yl)methanone (16)

[4-(3,5-Dimethoxyphenyl)piperazin-1-yl]-[1-(4-nitrophenyl)-5-trifluoromethyl-1H-pyrazol-4-yl]-methanone (17)

Biological Actions of the Compounds According to the Invention

The in-vitro testing on selected tumor models showed the following pharmacological activities.

### EXAMPLE 18

### Antiproliferative Action on Various Tumor Cell Lines

The substances according to the invention were investigated for their antiproliferative activity in a proliferation test on established tumor cell lines. The test used determines the cellular dehydrogenase activity and makes possible a determination of the cell vitality and indirectly the cell count. The cell lines used are the human cervical carcinoma cell line KB/HeLa, (ATCC CCL17), the ovarian adenocarcinoma cell line SKOV-3 (ATCC HTB77), the human glioblastoma cell line SF-268 (NCI 503138) and the lung carcinoma cell line NCI-H460 (NCI 503473). In addition, for the investigation of the cell cycle-specific action of the substance, an RKOp27 cell system was used (M. Schmidt et al. Oncogene 19(20):2423-9, 2000). RKO is a human colon carcinoma cell line, in which the cell cycle inhibitor p₂₇^{kip1} induced by means of the ecdysone expression system is expressed and can be led to a cell cycle arrest specifically in G2. A nonspecifically acting substance inhibits the proliferation independently of weather the RKO cell is or is not arrested in G1 or G2. Cell cycle-specific substances such as, for example, tubulin inhibitors are, however, only cytotoxic if cells are not arrested and the cell cycle is passed through. In table 1, the cytotoxic and/or growth-inhibiting activities of the compound described with/without expression of p27^{kip1} are shown. The compounds tested showed no cytotoxic activities in the induced state of p27^{kip1}. The results show a very potent inhibition of the proliferation of selected tumor cell lines by the compounds according to the invention.

TABLE 1
Inhibition of proliferation of selected compounds in the XTT
cytotoxicity test on human tumor cell lines

| | XTT proliferation assay, EC50 in μg/ml | | | | | |
|---|---|---|---|---|---|---|
| Com- | KB/ | | | | | |
| pound | Hela | SKOV3 | SF-268 | NCI-H460 | RKOP27 | RKOP27 |
| 1 | 0.555 | 0.400 | 0.309 | 0.312 | 0.208 | >3.16 |
| 2 | 2.592 | 0.585 | 0.939 | 0.886 | 0.326 | >3.16 |
| 3 | 4.322 | 0.397 | 0.478 | 0.853 | 0.726 | >3.16 |
| 5 | 1.212 | 0.496 | 0.474 | 0.348 | 0.250 | >3.16 |
| 7 | 2.710 | 1.010 | n.c. | 1.540 | 1.200 | >3.16 |
| 8 | 0.929 | 0.287 | 0.775 | 0.439 | 0.291 | >3.16 |
| 9 | 0.613 | 0.341 | 0.692 | 0.427 | 0.217 | >3.16 |
| 10 | 0.166 | 0.082 | 0.094 | 0.085 | 0.082 | >3.16 |
| 12 | 0.080 | 0.029 | 0.075 | 0.064 | 0.058 | >3.16 |
| 13 | 0.628 | 0.293 | 0.408 | 0.29 | 0.193 | >3.16 |
| 14 | 0.012 | 0.008 | 0.009 | 0.005 | 0.006 | >3.16 |
| 15 | 0.040 | 0.018 | 0.036 | 0.024 | 0.022 | >3.16 |
| 16 | 0.147 | 0.082 | 0.100 | 0.087 | 0.064 | >3.16 |

n.c.: not carried out

### EXAMPLE 19

### Inhibition of the Polymerization of Tubulin

Selected substances were tested for inhibition of the polymerization of bovine tubulin in an in-vitro test. In this test, tubulin purified by cycles of polymerization and depolymerization is employed, which is polymerized by addition of GTP and warming. In Table 2, the EC₅₀ values of the inhibition of polymerization of tubulin with 30% associated proteins (MAPs) and of MAP-free tubulin are indicated. The results show a good to very good inhibitory action of the substances according to the invention on the polymerization of tubulin.

TABLE 2
Inhibition of tubulin polymerization.
Average value from two independent experiments.

Inhibition of tubulin polymerization,
EC50 in μg/ml

| Compound | with 30% MAPs | without MAPs |
|---|---|---|
| 1 | 0.86 | 1.36 |
| 3 | 4.77 | n.c. |
| 8 | 5.66 | n.c. |
| 10 | 1.18 | n.c. |
| 12 | 1.16 | 1.71 |
| 13 | 0.73 | n.c. |
| 14 | 0.46 | n.c. |
| 15 | 0.88 | n.c. |
| 16 | 4.20 | n.c. |

n.c.: not carried out

Description of the Methods Used

XTT Test for Cellular Dehydrogenase Activity

The adherently growing tumor cell lines KB/HeLa, SKOV-3, SF-268 and NCI-H460 were cultured under standard conditions in a fumigation incubator at 37° C., 5% CO₂ and 95% atmospheric humidity. On experimental day 1, the cells are detached using trypsin/EDTA and pelleted by centrifugation. Subsequently, the cell pellet is resuspended in the respective culture medium at the corresponding cell count and reacted in a 96-well microtiter plate. The plates are then cultured overnight in the fumigation incubator. The test substances are prepared as 1 mg/ml stock solutions in DMSO and diluted to the appropriate concentrations on experimental day 2 using culture medium. The substances in culture medium are then added to the cells and incubated in the fumigation incubator for 45h. As a control, cells which are not treated with test substance are used. For the XTT assay, 1 mg/ml of XTT (sodium 3′-[1-(phenylaminocarbonyl)-3,4-tetrazolium]-bis(4-methoxy-6-nitro)benzenesulfonic acid) is dissolved in RPMI-1640 medium without Phenol Red. Additionally, a 0.383 mg/ml PMS (N-methyldibenzopyrazine methylsulfate) solution in phosphate-buffered saline solution (PBS) is prepared. On experimental day 4, 75 μl/well of XTT-PMS mixture is pipetted onto the cell plates which in the meantime have been incubated with the test substances for 45 h. For this, shortly before use, the XTT solution is mixed with the PMS solution in the ratio 50:1 (vol:vol). The cell plates are then incubated in the fumigation incubator for a further 3h and the optical density (OD₄₉₀ₙₘ) is determined in a photometer. By means of the OD₄₉₀ₙₘ determined, the percentage inhibition is calculated relative to the control and plotted semilogarithmically in the form of a concentration-action curve. The EC₅₀ is calculated by means of a regression analysis from the concentration-action curve using the program Graphpad Prism.

Cell Cycle Analysis by Means of the RKOp27 Model

The assay is carried out in 96-well plates. By inducible expression of p27^{kip1}, the cells are completely arrested in growth, but do not die. By comparison of the activity on induced and noninduced cells, conclusions on the mechanism of action (cell cycle specificity) of the therapeutics can be drawn. Noninduced cells are inoculated in approximately three-fold higher cell count, since division no longer takes place during the assay in comparison with uninduced cells (20000 cells/well induced, 6250 cells/well not induced). The controls are untreated cells (+/−induction). The induction is carried out with 3 μM muristerone A On the 1st day, the cells are exposed (+/− muristerone A) and incubated at 37° C. for 24h. On day 2, the test substance is added (control DMSO) and incubation is continued at 37° C. for a further 45 h before a standard XTT assay is carried out.

Tubulin Polymerization Assay

The assay is carried out based on the method of Bollag et. al. Lyophilized bovine tubulin (cytoskeleton, ML113 tubulin 30% MAPs, TL238 tubulin MAP free) is dissolved in a concentration of 2 mg/ml (ML113 in 80 mM PIPES, 0.5 mM EGTA, 2 mM MgCl₂, pH6.9, 1 mM GTP) or 5 mg/ml (TL238 in 80 mM PIPES, 1 mM EGTA, 0.5 mM MgCl₂, 20% (v:v) glycerol pH 6.9, 1 mM GTP). The test substances are diluted in 10% DMSO (v:v) and 5 μl of the dilutions are transferred to a 96-well microtiter plate (Nunc, half area plate). After addition of 45 μl of the tubulin solution, the polymerization is determined at 340 nm in a Spectramax 190 microtiter plate reader (Molecular devices) by means of a kinetics program at 30 sec intervals over a period of 20 min. The resulting area under curve values are used for the calculation of the inhibition with respect to the untreated control and plotted semilogarithmically in the form of a concentration-action curve. The EC₅₀ is calculated by means of a regression analysis from the concentration-action curve using the program Graphpad Prism.

### EXAMPLES OF PHARMACEUTICAL ADMINISTRATION FORMS

Example I
Tablet containing 50 mg of active compound

| Composition: | | |
|---|---|---|
| (1) Active compound | 50.0 | mg |
| (2) Lactose | 98.0 | mg |
| (3) Cornstarch | 50.0 | mg |
| (4) Polyvinylpyrrolidone | 15.0 | mg |
| (5) Magnesium stearate | 2.0 | mg |
| Total: | 215.0 | mg |

Preparation:

(1), (2) and (3) are mixed and granulated with an aqueous solution of (4) granuliert (5) is admixed to the dried granules. Tablets are pressed from this mixture.

Example II
Capsule containing 50 mg of active compound

| Composition: | | |
|---|---|---|
| (1) Active compound | 50.0 | mg |
| (2) Cornstarch, dried | 58.0 | mg |
| (3) Lactose, powdered | 50.0 | mg |
| (4) Magnesium stearate | 2.0 | mg |
| Total: | 160.0 | mg |

Preparation:

(1) is triturated with (3). This trituration is added to the mixture of (2) and (4) with intensive, mixing, This powder mixture is filled into hard gelatine capsules size 3 on a capsule filling machine.

1. A novel aryl- or heteroaryl-substituted piperazinylcarbonyl compound as in the general formula (1), 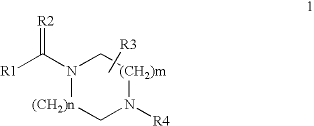
where the substituents have the following meaning:
R1: fluoren-9-one, isoxazole, cinnoline, isothiazole, isoquinoline, 9H-fluorene, 9H-xanthene and 1H-pyrazole,
where the bonding can take place via any desired and possible ring member of the heteroaryl or aryl radical and the aromatics and heteroaromatics can be mono- or polysubstituted or unsubstituted,
R2: O, S;
R3: represents one or up to 16 substituents selected from the group: H, unsubstituted or substituted alkyl, halogen, COOH, CONH₂,
where the substituents can be arranged vicinally or geminally on the heterocycle;
R4: unsubstituted or substituted aryl, unsubstituted or substituted heteroaryl, unsubstituted or substituted alkylaryl, unsubstituted or substituted alkylhetaryl;
m, n: 0-3.

2. An aryl- or heteroarylcarbonylpiperazine compound of the general formula (1) as claimed in claim 1, in which
“halogen” comprises the halogen atoms fluorine, chlorine, bromine and iodine,
“metal” comprises metal ions such as sodium, potassium, lithium, magnesium, calcium, zinc and manganese ions,
“alkyl” comprises acyclic saturated or unsaturated hydrocarbon radicals, having 1 to 20 C atoms, which can be branched or straight-chain and unsubstituted or mono- or polysubstituted, alkenyls having at least one C—C double bond and alkynyls at least one C—C triple bond,
“cycloalkyl” comprises cyclic hydrocarbons, having 3-12 carbon atoms, which can be saturated or unsaturated, unsubstituted or substituted, whose binding to the compounds of the general formula (1) can take place via any desired and possible ring member of the cycloalkyl radical and the cycloalkyl radical can also be part of a bi- or polycyclic system,
“heterocyclyl” stands for a 3-, 4-, 5-, 6-, 7- or 8-membered cyclic organic radical, which is unsubstituted or mono- or polysubstituted, saturated or unsaturated, but not aromatic, which contains at least 1, optionally 2, 3, 4 or 5 heteroatoms, preferably nitrogen, oxygen and sulfur, where the heteroatoms are identical or different and whose bonding to the compounds of the general formula (1) can take place via any desired and possible ring member of the heterocyclyl radical, where the heterocycle can also be part of a bi- or polycyclic system,
“aryl” denotes aromatic hydrocarbons, which are unsubstituted or mono- or polysubstituted, inter alia phenyls, naphthyls and anthracenyls, whose radicals can also be fused to further saturated, (partially) unsaturated or aromatic ring systems and whose bonding to the compounds of the general formula (1) can take place via any desired and possible ring member of the aryl radical,
“heteroaryl” stands for a 5-, 6- or 7-membered cyclic aromatic radical, which is unsubstituted or mono- or polysubstituted, identically or differently, which contains at least 1, optionally also 2, 3, 4 or 5 heteroatoms, preferably nitrogen, oxygen and sulfur, where the heteroatoms are identical or different and whose bonding to the compounds of the general formula (1) can take place via any desired and possible ring member of the heteroaryl radical, where the heterocycle can also be part of a bi- or polycyclic system,
“alkylcycloalkyl”, “alkylheterocyclyl”, “alkylaryl” or “alkylheteroaryl” have the meanings defined for alkyl, cycloalkyl, heterocyclyl, aryl and heteroaryl and the cycloalkyl, heterocyclyl, aryl or heteroaryl radical is bonded to the compounds of the general formula (1) via a C₁₋₈-alkyl group.
“substituted” in connection with “alkyl”, “alkenyl” and “alkynyl” can denote the substitution of a hydrogen radical by F, Cl, Br, I, CN, NH₂, NH-alkyl, NH-cycloalkyl, NH-aryl, NH-heteroaryl, NH-alkylaryl, NH-alkylheteroaryl, NH-heterocyclyl, NH-alkyl-OH, N(alkyl)₂, N(alkylaryl)₂, N(alkylheteroaryl)₂, N(heterocyclyl)₂, N(alkyl-OH)₂, NO, NO₂, SH, S-alkyl, S-cycloalkyl, S-aryl, S-heteroaryl, S-alkylaryl, S-alkylheteroaryl, S-heterocyclyl, S-alkyl-OH, S-alkyl-SH, S-alkyl, S—S-cycloalkyl, S—S-aryl, S—S-heteroaryl, S—S-alkylaryl, S—S-alkylheteroaryl, S—S-heterocyclyl, S—S-alkyl-OH, S—S-alkyl-SH, S—S-alkyl-C(O)—NH-heterocyclyl, OH, O-alkyl, O-cycloalkyl, O-alkylcycloalkyl, O-aryl, O-heteroaryl, O-alkylaryl, O-alkylheteroaryl, O-heterocyclyl, O-alkylheterocyclyl, O-alkyl-OH, O-alkyl-O-alkyl, O—SO₂—N(alkyl)₂, O—SO₂—OH, O—SO₂—O-alkyl, O—SO₂—O-cycloalkyl, O—SO₂—O-heterocycloalkyl, O—SO₂—O-alkylcycloalkyl, O—SO₂—O-alklheterocycloalkyl, O—SO₂—O-aryl, O—SO₂—O-heteroaryl, O—SO₂—O-alkylaryl, O—SO₂—O-alkylheteroaryl, O—SO₂-alkyl, O—SO₂-cycloalkyl, O—SO₂-heterocycloalkyl, O—SO₂-alkylcycloalkyl, O—SO₂-alkylheterocycloalkyl, O—SO₂-aryl, O—SO₂-heteroaryl, O—SO₂-alkylaryl, O—SO₂-alkylheteroaryl, O—C(O)-alkyl, O—C(O)-cycloalkyl, O—C(O)-heterocycloalkyl, O—C(O)-alkylcycloalkyl, O—C(O)-alkylheterocycloalkyl, O—C(O)-aryl, O—C(O)-heteroaryl, O—C(O)-alkylaryl, O—C(O)-alkylheteroaryl, O—C(O)O-alkyl, O—C(O)O-cycloalkyl, O—C(O)O-heterocycloalkyl, O—C(O)O-alkylcycloalkyl, O—C(O)O-alkylheterocycloalkyl, O—C(O)O-aryl, O—C(O)O-heteroaryl, O—C(O)O-alkylaryl, O—C(O)O-alkylheteroaryl, O—C(O)NH-alkyl, O—C(O)NH-cycloalkyl, O—C(O)NH-heterocycloalkyl, O—C(O)NH-alkylcycloalkyl, O—C(O)NH-alkylheterocycloalkyl, O—C(O)NH-aryl, O—C(O)NH-heteroaryl, O—C(O)NH-alkylaryl, O—C(O)NH-alkylheteroaryl, O—C(O)N(alkyl)₂, O—C(O)N(cycloalkyl)₂, O—C(O)N(heterocycloalkyl)₂, O—C(O)N(alkylcycloalkyl)₂, O—C(O)N(alkylheterocycloalkyl)₂, O—C(O)N(aryl)₂, O—C(O)N(heteroaryl)₂, O—C(O)N(alkylaryl)₂, O—C(O)N(alkylheteroaryl)₂, O—P(O)(OH)₂, O—P(O)(O-metal)₂, O—P(O)(O-alkyl)₂, O—P(O)(O-cycloalkyl)₂, O—P(O)(O-aryl)₂, O—P(O)(O-heteroaryl)₂, O—P(O)(O-alkylaryl)₂, O—P(O)(O-alkylheteroaryl)₂, O—P(O)(N-alkyl)₂(N-alkyl)₂, O—P(O)(N-cycloalkyl)₂(N-cycloalkyl)₂, O—P(O)(N-heterocycloalkyl)₂(N-heterocycloalkyl)₂, O—P(O)(N-heteroaryl)₂(N-heteroaryl)₂, O—P(O)(N-alkylaryl)₂(N-alkylaryl)₂, O—P(O)(N-alkylheteroaryl)₂(N-alkylheteroaryl)₂, CHO, C(O)-alkyl, C(S)-alkyl, C(O)-aryl, C(S)-aryl, C(O)-alkylaryl, C(S)-alkylaryl, C(O)-heterocyclyl, C(O)-heteroaryl, C(O)-alkylheteroaryl, C(S)-heterocyclyl, CO₂H, CO₂-alkyl, CO₂-cyclyl, CO₂-heterocyclyl, CO₂-aryl, CO₂-heteroaryl, CO₂-alkylaryl, C(O)—NH₂, C(O)NH-alkyl, C(O)NH-aryl, C(O)NH-heterocyclyl, C(O)NH-alkylheterocyclyl, C(O)N(alkyl)₂, C(O)N(alkylaryl)₂, C(O)N(alkylheteroaryl)₂, C(O)N(heterocyclyl)₂, SO-alkyl, SO₂-alkyl, SO₂-aryl, SO₂-alkylaryl, SO₂-heteroaryl, SO₂-alkylheterocyclyl SO₂NH₂, SO₃H, CF₃, CHO, CHS, alkyl, cycloalkyl, aryl, alkylaryl, heteroaryl, alkylheterocyclyl and/or heterocyclyl, where in the case of polysubstituted radicals these can be polysubstituted either on different or on identical atoms and the polysubstitution can take place with the same or different substituents,
“substituted” in connection with aryl, heterocyclyl, heteroaryl, alkylaryl and cycloalkyl can mean the substitution of one or more hydrogen atoms of the ring system by F, Cl, Br, I, CN, NH₂, NH-alkyl; NH-aryl, NH-heteroaryl, NH-alkylaryl, NH-alkylheteroaryl, NH-heterocyclyl, NH-alkyl-OH, N(alkyl)₂, NC(O)alkyl, N(alkylaryl)₂, N(alkylheteroaryl)₂, N(heterocyclyl)₂, N(alkyl-OH)₂, NO, NO₂, SH, S-alkyl, S-aryl, S-heteroaryl, S-alkylaryl, S-alkylheteroaryl, S-heterocyclyl, S-alkyl-OH, S-alkyl-SH, OH, O-alkyl, O-cycloalkyl, O-alkylcycloalkyl, O-aryl, O-heteroaryl, O-alkylaryl, O-alkylheteroaryl, O-heterocyclyl, O-alkylheterocyclyl, O-alkyl-OH, O-alkyl-O-alkyl; O—SO₂—N(alkyl)₂, O—SO₂—OH, O—SO₂—O-alkyl, O—SO₂—O-cycloalkyl, O—SO₂—O-heterocycloalkyl, O—SO₂—O-alkylcycloalkyl, O—SO₂—O-alkylheterocycloalkyl, O—SO₂—O-aryl, O—SO₂—O-heteroaryl, O—SO₂—O-alkylaryl, O—SO₂—O-alkylheteroaryl, O—SO₂-alkyl, O—SO₂-cycloalkyl, O—SO₂-heterocycloalkyl, O—SO₂-alkylcycloalkyl, O—SO₂-alkylheterocycloalkyl, O—SO₂-aryl, O—SO₂-heteroaryl, O—SO₂-alkylaryl, O—SO₂alkylheteroaryl, O—C(O)-alkyl, O—C(O)-cycloalkyl, O—C(O)-heterocycloalkyl, O—C(O)-alkylcycloalkyl, O—C(O)-alkylheterocycloalkyl, O—C(O)-aryl, O—C(O)-heteroaryl, O—C(O)-alkylaryl, O—C(O)-alkylheteroaryl, O—C(O)O-alkyl, O—C(O)O-cycloalkyl, O—C(O)O-heterocycloalkyl, O—C(O)O-alkylcycloalkyl, O—C(O)O—alkylheterocycloalkyl, O—C(O)O-aryl, O—C(O)O-heteroaryl, O—C(O)O-alkylaryl, O—C(O)O-alkylheteroaryl, O—C(O)NH-alkyl, O—C(O)NH-cycloalkyl, O—C(O)NH-heterocycloalkyl, O—C(O)NH-alkylcycloalkyl, O—C(O)NH-alkylheterocycloalkyl, O—C(O)NH-aryl, O—C(O)NH-heteroaryl, O—C(O)NH-alkylaryl, O—C(O)NH-alkylheteroaryl, O—C(O)N(alkyl)₂, O—C(O)N(cycloalkyl)₂, O—C(O)N(heterocycloalkyl)₂, O—C(O)N(alkylcycloalkyl)₂, O—C(O)N(alkylheterocycloalkyl)₂, O—C(O)N(aryl)₂, O—C(O)N(heteroaryl)₂, O—C(O)N(alkylaryl)₂, O—C(O)N(alkylheteroaryl)₂, O—P(O)(OH)₂, O—P(O)(O-metal)₂, O—P(O)(O-alkyl)₂, O—P(O)(O-cycloalkyl₂, O—P(O)(O-aryl)₂, O—P(O)(O-heteroaryl)₂, O—P(O)(O-alkylaryl)₂, O—P(O)(O-alkylheteroaryl)₂, O—P(O)(N-alkyl)₂(N-alkyl)₂, O—P(O)(N-cycloalkyl)₂(N-cycloalkyl)₂, O—P(O)(N-heterocycloalkyl)₂(N-heterocycloalkyl)₂ O—P(O)(N-aryl)₂(N-aryl)₂, O—P(O)(N-heteroaryl)₂(N-heteroaryl)₂, O—P(p)(N-alkylaryl)₂(N-alkylaryl)₂, O—P(O)(N-alkylheteroaryl)₂(N-alkylheteroaryl)₂, CHO, C(O)-alkyl, C(S)-alkyl, C(O)-aryl, C(S)-aryl, C(O)-alkylaryl, C(S)-alkylaryl, C(O)-heterocyclyl, C(S)-heterocyclyl, CO₂H, CO₂-alkyl, CO₂-alkylaryl, C(O)—NH₂, C(O)NH-alkyl, C(O)NH-aryl, C(O)NH-heterocyclyl, C(O)N(alkyl)₂, C(O)N(alkylaryl)₂, C(O)N(alkylheteroaryl)₂, C(O)N(heterocyclyl)₂, SO-alkyl, SO₂-alkyl, SO₂-aryl, SO₂-alkylaryl, SO₂-heteroaryl, SO₂-alkylheteroaryl, SO₂NH₂, SO₃H, CF₃, CHO, CHS, alkyl, cycloalkyl, aryl alkylaryl, heteroaryl, alkylheterocyclyl and/or heterocyclyl, where the substituents are identical or different and can occur in any desired and possible position of the aryl, heterocyclyl, heteroaryl, alkylaryl and cycloalkyl radical and where polysubstituted radicals can be polysubstituted with the same or with different substituents, either on different or on identical atoms.

3. An aryl- or heteroarylcarbonylpiperazine compound of the general formula (1) as claimed in claims 1 and 2, wherein the alkyl radical can be methyl, ethyl, n-propyl, 2-propyl, n-butyl, sec.-butyl, tert.-butyl, n-pentyl, iso-pentyl, neo-pentyl, n-hexyl, 2-hexyl, n-octyl, ethylenyl (vinyl), ethynyl, propenyl, (—CH₂CH═CH₂; —CH═CH—CH₃, —C(═CH₂)—CH₃), propynyl(—CH₂—C≡CH, —C≡C—CH₃), butenyl, butynyl, pentenyl, pentynyl, hexenyl, hexynyl, octenyl and octynyl.

4. An aryl- or heteroarylcarbonylpiperazine compound of the general formula (1) as claimed in claims 1 and 2, wherein the heterocyclyl radical can be tetrahydrofuryl, tetrahydropyranyl, pyrrolidinyl, piperidinyl, piperazinyl and morpholinyl.

5. An aryl- or heteroarylcarbonylpiperazine compound of the general formula (1) as claimed in claims 1 and 2, wherein the heteroaryl radical can be pyrrolyl, furyl, thienyl, thiazolyl, triazolyl, tetrazolyl, oxazolyl, isothiazolyl, isoxazolyl, pyrazolyl, imidazolyl, pyridinyl, pyrimidinyl, pyrazinyl, triazinyl, benzothiazolyl, indolyl, indolizinyl, quinolinyl, isoquinolinyl, cinnolinyl, quinazolinyl, quinoxalinyl, phthalazinyl, carbazolyl, phenazinyl, phenothiazinyl, purinyl, acridinyl, phenanthrinyl.

6. A compound of the general formula (1) as claimed in claims 1-5, wherein R₁, R₂, R₃, n and m have the abovementioned meanings and R₄ stands for phenyl, which is unsubstituted or substituted by one to five identical or different (C₁-C₆)-alkoxy groups, where adjacent oxygen atoms can also be linked by (C₁-C₂)-alkylene groups.

7. A compound of the general formula (1) as claimed in claims 1-5, wherein R, R₁, R₂, R₃, n and m have the abovementioned meanings and R₄ stands for 3,5-dimethoxyphenyl.

8. A compound of the general formula (1) as claimed in claims 1-5, wherein R, R₁, R₂, R₃, n and m have the abovementioned meanings and R₄ stands for 3-methoxyphenyl.

9. A physiologically tolerable salt of the compounds as in formula (1) as claimed in claims 1-8, comprised by neutralization of the basic compounds with inorganic and organic acids or neutralization of the acidic compounds with inorganic and organic bases, or its solvates and hydrates.

10. An aryl- or heteroarylcarbonylpiperazine compound of the general formula (1) as claimed in claims 1-9, having at least one asymmetric carbon atom, in the form of its racemates, in the form of the pure enantiomers and/or diastereomers or in the form of mixtures of these enantiomers and/or diastereomers or in the form of the tautomers.

11. A compound of the general formula (1), in particular one of the following compounds:
4-[4-(3,5-Dimethoxyphenyl)piperazine-1-carbonyl]fluoren-9-one (1)
4-[4-(6-methylpyridin-2-yl)piperazine-1-carbonyl]fluoren-9-one (2)
4-[4-(3-Hydroxyphenyl)piperazine-1-carbonyl]fluoren-9-one (3)
[4-(3,5-Dimethoxyphenyl)piperazin(1-yl]-(5-methyl-3-phenylisoxazol-4-yl)methanone (4)
cinnolin-4-yl-[4-(3,5-dimethylphenyl)piperazin-1-yl]methanone (5)
cinnolin-4-yl-[4-(6-methylpyridin-12-yl)piperazin-1-yl]methanone (6)
(3,5-Bis-methylsulfanylisothiazol-4-yl)-[4-(6-methylpyridin-2-yl)piperazin-1-yl]methanone (7)
[0.4-(3,5-Dimethoxyphenyl)piperazin-1-yl]-isoquinolin-1-ylmethanone (8)
[4-(3,5-Dimethoxyphenyl)piperazin-1-yl]-(9H-fluoren-1-yl)methanone (9)
(9H-Fluoren-9-yl)-[4-(3-methoxyphenyl)piperazin-1-yl]methanone (10)
(9H-Fluoren-1-yl)-[4-(3-methoxyphenyl)piperazin-1-yl]methanone (11)
[4-(3,5-Dimethoxyphenyl)piperazin-1-yl]-(9H-xanthen-9-yl)methanone (12)
[4-(3-methoxyphenyl)piperazin-1-yl]-(9H-xanthen-9-yl)methanone (13)
[4-(3-methoxyphenyl)piperazin-1-yl]-(2-phenyl-2H-pyrazol-3-yl)methanone (14)
[4-(6-methylpyridin-2-yl)piperazin-1-yl]-(2-phenyl-2H-pyrazol-3-yl)methanone (15)
[4-(3-Hydroxyphenyl)piperazin-1-yl]-(2-phenyl-2H-pyrazol-3-yl)methanone (16)
[4-(3,5-Dimethoxyphenyl)piperazin-1-yl]-[1-(4-nitrophenyl)-5-trifluoromethyl-1H-pyrazol-4-yl]methanone (17)

12. A process for the preparation of aryl- and heteroarylcarbonylpiperazine compounds as claimed in one of claims 1 to 11, which comprises reacting a carboxylic acid of the general formula 2, in which R1 and R2 have the abovementioned meanings and Y stands for a leaving group such as halogen, hydroxyl, (C1-C6)-alkoxy, preferably methoxy and ethoxy, —O-tosyl, —O-mesyl, tetrazolyl or imidazolyl, 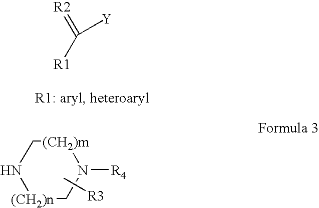
with an amine of the general formula 3, in which R₄, m and n have the above-mentioned meanings, optionally using a condensing agent and/or catalyst and diluents and auxiliaries with formation of the desired products.

13. The use of the aryl- and heteroarylcarbonylpiperazine compounds of the general formula (1) as claimed in one of claims 1 to 11 as therapeutic active compounds for the production of a medicament for the treatment of tumors in humans and in mammals.

14. A medicament for use in the treatment of tumors in humans and in mammals, comprising at least one compound of the general formula (1) as claimed in one of claims 1 to 11, preferably together with customary pharmaceutically tolerable excipients, additives and vehicles.

15. A medicament, comprising one or more compounds of the general formula (1) as claimed in one of claims 1-11 in addition to customary physiologically tolerable excipients, additives and vehicles.

16. A process for the production of a medicament as claimed in claim 15, which comprises processing one or more aryl- and heteroarylcarbonylpiperazine compounds of the general formula (1) as claimed in one of claims 1-11 with customary pharmaceutical vehicles and/or diluents or other excipients to give pharmaceutical preparations, or bringing them into a therapeutically administrable form.

17. A process for the treatment of benign and malignant tumors in humans and mammals, which comprises administering at least one compound of the general formula (1) as claimed in one of claims 1 to 11 to the human or mammal into a dose effective for tumor treatment.
